(19) United States
(12) Patent Application Publication
(10) Pub. No.: US 2024/0350285 A1
(43) Pub. Date: Oct. 24, 2024

(54) BIOMIMETIC DECODING OF SENSORIMOTOR INTENSION WITH ARTIFICIAL NEURAL NETWORKS

(71) Applicant: West Virginia University Board of Governors on Behalf of West Virginia University, Morgantown, WV (US)

(72) Inventors: Sergiy Yakovenko, Morgantown, WV (US); Serhii Bahdasariants, Morgantown, WV (US)

(21) Appl. No.: 18/686,027

(22) PCT Filed: Aug. 24, 2022
(86) PCT No.: PCT/US2022/075404

§ 371 Date: Feb. 23, 2024

Related U.S. Application Data
(60) Provisional application No. 63/236,667, filed on Aug. 24, 2021.

(51) Int. Cl. A61F 2/72; A61F 2/54; A61F 2/70; G06F 30/10

(52) U.S. Cl. CPC A61F 2/72; G06F 30/10; A61F 2/54; A61F 2002/704

(57) ABSTRACT

Various examples are provided related to biomimetics and use of ANNs for decoding and control. In one example. a method includes generating muscle model parameters by a musculoskeletal kinematic transformation implemented by a first artificial neural network. generating one or more physics engine parameters from a muscle model, and generating a physics engine transformation implemented by a second artificial neural network based at least in part upon the one or more physics engine parameters. The muscle model parameters can be based at least in part upon sensor inputs and the one or more physics engine parameters can be based at least in part the muscle model parameters. A sensorimotor mechanism can be controlled based on the physics engine transformation. In another example. a system for prosthetic control includes a plurality of sensors and processing circuitry configured to control a sensorimotor mechanism based upon the physics engine transformation.

### CROSS REFERENCE TO RELATED APPLICATIONS

This application claims priority to, and the benefit of, co-pending U.S. provisional application entitled “Biomimetic Decoding of Sensorimotor Intension with Artificial Neural Networks” having Ser. No. 63/236,667, filed Aug. 24, 2021, which is hereby incorporated by reference in its entirety.

### STATEMENT REGARDING FEDERALLY SPONSORED RESEARCH OR DEVELOPMENT

This invention was made with government support under W911NF-15-2-0016 awarded by Defense Advanced Research Projects Agency (DARPA). The Government has certain rights in the invention.

### BACKGROUND

Developing a fast and intuitive interface with a high-dimensional artificial limb is a challenging task solved increasingly with the help of pattern recognition algorithms. Typically, myoelectric direct and proportional control is used to decode motor intent from the recorded surface electromyography (EMG) and convert it into joint torques or furthermore positions of the powered prosthetic devices. This has been previously accomplished using ANN decoding algorithms decoding kinematics from EMG signals.

### SUMMARY

Aspects of the present disclosure are related to biomimetics and use of artificial neural networks (ANNs) for decoding and control. In one aspect, among others, a method comprises generating muscle model parameters by a musculoskeletal kinematic transformation implemented by a first artificial neural network, the muscle model parameters based at least in part upon sensor inputs; generating one or more physics engine parameters from a muscle model, the one or more physics engine parameters based at least in part on the muscle model parameters; and generating a physics engine transformation implemented by a second artificial neural network based at least in part upon the one or more physics engine parameters, the physics engine transformation representing segment dynamics and interactions with environment. The method can comprise controlling a sensorimotor mechanism based upon the physics engine transform.

In one or more aspects, the muscle model parameters can comprise muscle and joint parameters. The muscle and point parameters can comprise a plurality of muscle lengths and a plurality of moment arms. The physics engine parameters can comprise joint torque and/or neural activity. The sensor inputs can comprise surface electromyography signals. In various aspects, training datasets for the first artificial neural network of the musculoskeletal kinematic transformation can be generated using an approximation of musculoskeletal relationships. The first artificial neural network can be trained using a supervised learning approach. The first and second artificial neural networks can have a latency of less than 20 ms.

In another aspect, a system for prosthetic control comprises a plurality of sensors; and processing circuitry configured to control a sensorimotor mechanism based upon a physics engine transformation implemented by a second artificial neural network based at least in part upon one or more physics engine parameters generated from a muscle model, the one or more physics engine parameters based at least in part on muscle model parameters generated by a musculoskeletal kinematic transformation implemented by a first artificial neural network, the muscle model parameters based at least in part upon sensor inputs from the plurality of sensors. In one or more aspects, the plurality of sensors can comprise surface electromyography sensors. The physics engine parameters can comprise joint torque and/or neural activity. The muscle model parameters can comprise muscle and joint parameters. The muscle and point parameters can comprise muscle lengths and moment arms.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of motor intent decoding from muscle activity, in accordance with various embodiments of the present disclosure.

FIGS. 2A and 2B illustrate examples of forward and inverse representations of kinematic motor intent and expected kinetics calculation, respectively, in accordance with various embodiments of the present disclosure.

FIGS. 3A-3C illustrate examples of training and testing of ML models, in accordance with various embodiments of the present disclosure.

FIGS. 4A-4D illustrate examples of the relationship between model performance (RMSE) and the training dataset size for the ANN and LGB models, in accordance with various embodiments of the present disclosure.

FIGS. 5A-5D illustrate examples of distributions of errors in the prediction of muscle length and moment arms for the ANN and LGB models, in accordance with various embodiments of the present disclosure.

FIG. 6 illustrates an example of normalized absolute errors for muscle length and moment arms evaluated with the ANN, in accordance with various embodiments of the present disclosure.

FIG. 7 is a schematic diagram illustrating an example of processing circuitry that can be utilized in sensorimotor mechanisms, in accordance with various embodiments of the present disclosure.

### DETAILED DESCRIPTION

Disclosed herein are various examples related to biomimetic algorithms that utilize artificial neural networks (ANN). Human-machine interfaces that transform biological activity of muscles and nerves into the body segment movement can be utilized for intuitive wearables. There are no previous ANN solutions for dynamical systems that describe high-dimensional systems. For example, a human arm and hand is a 23 degree of freedom system. The disclosed solution uses mechanistic models of MS and PE. A hierarchical system can be used that parses the input-output problem into a series of biomimetic and distinct transformations that can be accurately mapped with ANNs. This can be implemented on dedicated hardware to provide real-time metrics and control in multiple human-machine interfaces such as, e.g., prosthetics. The solution offers the reduction of overall complexity for the training process, creates metrics of performance at the intermediate states within the process, and offers implementations that adapt to subject morphology. Reference will now be made in detail to the description of the embodiments as illustrated in the drawings, wherein like reference numbers indicate like parts throughout the several views.

Machine learning (ML) with artificial neural networks (ANN) is revolutionizing applications where recognition, cognition, and categorization abilities previously utilized direct human involvement. While the initial focus was on the substitution of human operators, for example, in driving a vehicle, classifying media, or for radiological diagnostics of benign or malignant mass in mammography, the reach of ANN can be extended to the exploration of sensorimotor mechanisms that could potentially work in the closed-loop systems with human operator. The structural and functional complexity of this system, which is distributed across multiple neural and mechanical pathways and has high-dimensional computations for the segmented body control, offers a unique challenge and opportunities for this approach.

One of the opportunities lies within the innate ability of ANNs to absorb and classify a high volume of multidimensional input-output relationships. This is not dissimilar to biological processing responsible for coordinated spatiotemporal action of multiple muscles generating movement. Yet another extreme expression of the neural processing complexity and efficiency is the brain's ability to solve the Bernsteinian degrees of freedom problem where the same motor goal of body control can be accomplished with different kinematic solutions. ML methods based on ANNs can potentially resolve or, at least, identify targets for the long-standing theoretical challenge that can provide insight to the current theories of neural processing and has multiple practical human-machine applications, e.g., in advanced prosthetics.

The intuitive control utilizes an additional transformation based on the representation of the controlled device and its neural control. The failure to account for the dynamics of prosthesis would lead to direct kinematic errors. The failure to recognize the biological strategies in solving limb dynamics would reduce robustness and intuitiveness of control even when the control of mechanical devices is perfectly tuned. The latter would occur because the interlimb inertial dynamics is encoded within neural commands even when limb dynamics changes. For example, mechanical shoulder immobilization does not abolish the stabilizing shoulder muscle activity during elbow movement. The expected musculoskeletal dynamics persists within neural commands months and years after the acute stage of limb trauma and amputations. The successful use of these commands for prosthetic control would theoretically require the representation of pre-trauma musculoskeletal and segmental limb dynamics to account for the dynamics encoded within neural control signals.

Relatively few studies applied ML techniques to musculoskeletal dynamics. The early applications of simple feed-forward ANNs allowed mapping of an average locomotor pattern of 16 EMGs to hip, knee, and ankle joint angles and moments. Even though the muscle paths and muscle force generation were not simulated in this disclosure, the decoding was demonstrated with low errors. The changes in the locomotor pattern at slow and fast speeds can also be generalized by a simple ANN with supervised learning via backpropagation. Furthermore, the mapping can be done not only between the locomotor activation patterns, but also with the muscle forces; albeit, the accurate predictions within trials have not been demonstrated. Yet, similar type of statistical mapping, admittedly, can be expressed with standard dimensionality reduction techniques with high precision and low computational cost, e.g., principal component analysis (PCA).

Multiple methodological variations have since been developed and applied to solve musculoskeletal problems. Notably recurrent ANNs were used to predict elbow torques from EMGs and showed the benefit of taking into account kinematic inputs, joint angle and velocity. A combination of convolutional and recurrent ANNs can accurately and robustly map from the time-frequency frames of multi-channel EMG to limb movement. Purely statistical learning of the musculoskeletal transformation from posture to the control inputs has been also demonstrated with multiple hybrid ANN methods for a 7 DOF robotic arm with artificial muscles. The musculoskeletal transformation was learnt from an input-output dataset. While the tracking of a two DOF arm was achieved, the control of both robot and its simulation resulted in large tracking errors. The high-dimensional control remains a challenge.

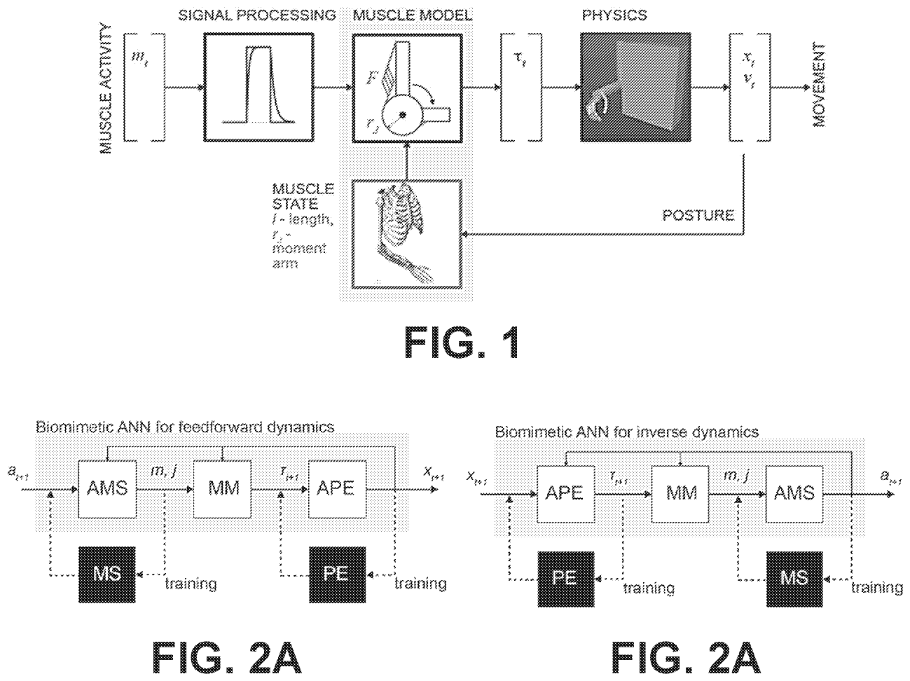

While data-driven mapping with ANNs or using PCA and other statistical classification alternatives are robust, these methods do not generally capture the mechanistic relationships. Intrinsic muscle properties and musculoskeletal organization contribute to the muscle force generation, and these mechanistic details may assist in the reconstruction of relationships between neural commands and generated movement. FIG. 1 schematically illustrates the general concept of motor intent decoding from muscle activity. The schematic illustrates an example of the transformation of EMG inputs through signal processing and musculoskeletal relationships (muscle model) into estimated torques that actuate limbs to generate movement by solving the equations of motion (physics). Limb posture modifies nonlinear muscle force-length-velocity relationship and torques.

The problem of learning the musculoskeletal dynamics (MSD) can be addressed with several ML techniques that may generate a computationally efficient solution. MSD requires high-dimensional transformations of posture into muscle moment arms and length, which are the essential variables in the calculation of generated muscle forces. The Hill-type muscle model can then allow the posture-dependent force-length-velocity dependency (Muscle Model) to be defined and next the muscle and joint torques computed. The remaining step for the generation of movement can be the simulation of equations of motion by using a physics engine or its approximation. An important constraint for the accuracy of these computations is the loop latency that limits the computational stability of integration. The trade-off between accuracy and latency can be achieved using methods similar to least-squared approximation, for example, used for the inverse dynamics computations. Thus, the goal for real-time biomechanics is to implement a method with high accuracy and low computational cost (low latency) of musculoskeletal transformations.

In this disclosure, estimating muscle moment arms and their muscle lengths can be solved from joint angles with two ML approaches. An arm and hand model can be used to generate input-output kinematic datasets, where joint angles were the input and the muscle length and moment arms were the output, and presented the comparative validation and performance metrics for the two solutions. The results of this disclosure develop the potential for mechanistic ML approaches that utilize the musculoskeletal transformation for online control problems.

A hierarchical system that parses the input-output problem into a series of biomimetic and distinct transformations that can be accurately mapped with ANNs is proposed. The transformation of time-series from neural activity (a) to body segment posture (x) is then the input-output relationship:

$$transformation ( in ) = out or in \to transformation ( ) \to out ; \qquad ( 1 )$$

$$Body ( a ( t + 1 ) ) = x ( t + 1 ) . \qquad ( 2 )$$

The function Body( ) is composed of a musculoskeletal kinematic transformation (MS) that provides muscle and joint parameters (m, j) to the muscle model (MM). The output of MM is joint torque (tau) which can be further evaluated with a physics engine (PE) solving the equations of motion for body segments. The transformation can then be expanded as the serial evaluation of the following functions:

$$Body ( ) = \{ a ( t + 1 ) , x ( t ) \} \to MS ( x ( t ) ) \to MM ( a , m , j ) \to PE ( tau , x ( t ) ) \to x ( t + 1 ) \} . \qquad ( 3 )$$

In the proposed solution, musculoskeletal transformation (MS) and body dynamics, which is often solved with physical engines (PE), are represented with ANN models—AMS and APE, respectively. These models are evaluated serially to map human motor intent to desired kinematics and vise versa. The AMS model can be trained to infer kinematics from the subject-specific moment arm and muscle lengths measurements, previously approximated with the power term polynomials. In turn, the APE can be trained to map between the joint torques and joint angles that can be further converted to body kinematics. The organization of trained networks for AMS and APE can be fine-tuned to the intended dynamics within these calculations. For example, the AMS can be a kinematic Jacobian that can be approximated with the power term polynomials, and, consequently, relatively shallow and standard ANNs can be employed. This APE can be expressed as a neural network mimicking Euler's numerical integration method, utilizing the recurrent inputs of kinematics within the transformation. FIGS. 2A and 2B schematically illustrate examples of forward and inverse representations for the calculation of kinematic motor intent and expected kinetics, respectively. These parsed computations can reduce the task of training by about 3 orders of magnitude.

The problem posed in the generic form of Eq. (2) can suffer from the exponential increase in complexity as the function of parametric dimensionality, also known as the curse of dimensionality. The description of the dataset needed to estimate the input-output relationships is related to the number of degrees of freedom (DOF) and meaningful samples for each DOF. The data organized as a grid increases exponentially with the number of DOFs, e.g., 5×5×5=5³ describes 3 DOFs sampled with 5 points. Some single muscles, for example the thumb muscles, span 6 DOFs and require 5^{6=15,625} points. For a system with 23 DOFs like the human arm and hand, the musculoskeletal structural complexity is independent of the temporal segmental dynamics. The independence of variables is generally captured by the multiplicative relationship. Then, the solution using the serial computations of Eq. (3) can decrease the dimensionality problem by about three orders of magnitude, or 1.5×10³ times. The savings would be expected to be even larger for systems attempting to solve the full body dynamics with about 100 degrees of freedom. This solution can practically enable training and re-training of models deployed on hardware such as, for example, dedicated Neural Engine chips from Apple, Inc. These computations can be subject specific and provide device control and performance metrics for users.

### Materials & Methods

Musculoskeletal Polynomial Model for Generation of Training and Testing

Datasets. A method of autogenerated polynomial models was previously developed. In these polynomials, the composition of terms can be expanded using objective information measurements, e.g., the corrected Akaike Information Criterion. In brief, the posture-dependent musculotendon actuator length and joint moment arms for each muscle in the upper-limb model can be accurately approximated using the selection of up to 5th power polynomial terms, where muscle length and moment arms are connected through a partial derivative of the muscle length in local coordinates corresponding to limb posture. Overall, the 18 DOF model of the human arm and hand is actuated by 33 muscles, each spanning about 3 DOFs and up to 6 DOFs for thumb muscles. Thus, each actuator is represented by a set of one length and about 3 moment arm-posture polynomials. The costly calculation of geometrical transformations may be bypassed with high-quality approximations. The high-fidelity of these approximations have been previously demonstrated with kinematic errors below 1%. In biomechanics, the errors of 1-5° in joint angles are expected from flaws in the observations in motion capture, and errors of 2° and less are not meaningful in the clinical context. Thus, the errors below 1% of joint range of motion are negligible. These polynomial models of muscle posture-dependent state can be used to develop an ANN-based approximation method for the musculoskeletal dynamics in this disclosure.

Training, Validation, and Testing Datasets. Training, validation, and testing datasets for the assessment of model performance were generated by the musculoskeletal polynomial model, which was used as a reference. Input-output relationships were extracted randomly with uniform distribution where the inputs were 18 DOF vectors of joint angles and the outputs were 33 length vectors and 99 moment arm vectors. An average muscle crosses 3 DOFs and has, consequently, 3 moment arm relationships on average. A supervised learning approach was used for training the ML models. The training dataset was used for two tasks, tuning the model hyper-parameters and model training, to maximize the model performance in replicating the desired outputs with given inputs.

The testing dataset contained about 5% of all data (5×10⁴ samples). The remaining about 95% were divided into the training dataset (80%, 8×10⁵ samples) and the validation dataset (20%, 2×10⁵ samples). The validation dataset was used to prevent overfitting, i.e., higher performance on the training data as compared to that on the validation data. These datasets were similarly used for training ANN and LGB models, described below. Overall, the training time was about 15 times longer for ANN then for LGB models. The training of all ANN and LGB models on the standard hardware took about 3.5 days.

Metrics. The performance of the trained models was further evaluated with the testing dataset, which was not used during the training procedure. The same error tolerances were expected to be reached as in previous polynomial fitting method studies. Consequently, the same normalization of lengths and moment arms was used as in previous work. The RMSE values were calculated as the absolute difference between reference and predicted muscle length values. To normalize the results, each reference and predicted length value can be divided by the muscle length range respectively:

$${RMSE}_{L} = \frac{1}{m} {\sum }_{l = 1}^{m} \frac{1}{n} {\sum }_{i = 1}^{n} \sqrt{{( \frac{{x}_{r , l , i} - {x}_{p , l , i}}{L {max}_{l} - L {min}_{l}} )}^{2}} , \qquad ( 4 )$$

where m is the number of muscles (e.g., m=33), n is the number of test samples, x_{r,l,i} and x_{p,l,i} are reference and predicted length values, respectively, Lmaxₗ and Lminₗ are the maximum and minimum values over the full range of lth muscle length.

Similarly, the RMSE of moment arms was calculated as the absolute difference between reference and predicted values, which were normalized to the moment arm maximum (Mmaxₗ):

$${RMSE}_{MA} = \frac{1}{m} {\sum }_{l = 1}^{m} \frac{1}{n} {\sum }_{i = 1}^{n} \sqrt{{( \frac{{x}_{r , l , i} - {x}_{p , l , i}}{M {max}_{l}} )}^{2}} , \qquad ( 5 )$$

where x_{r,l,i} and x_{p,l,i} are reference and predicted values, m is the number of moment arms (e.g., m=99), and n is the number of test samples.

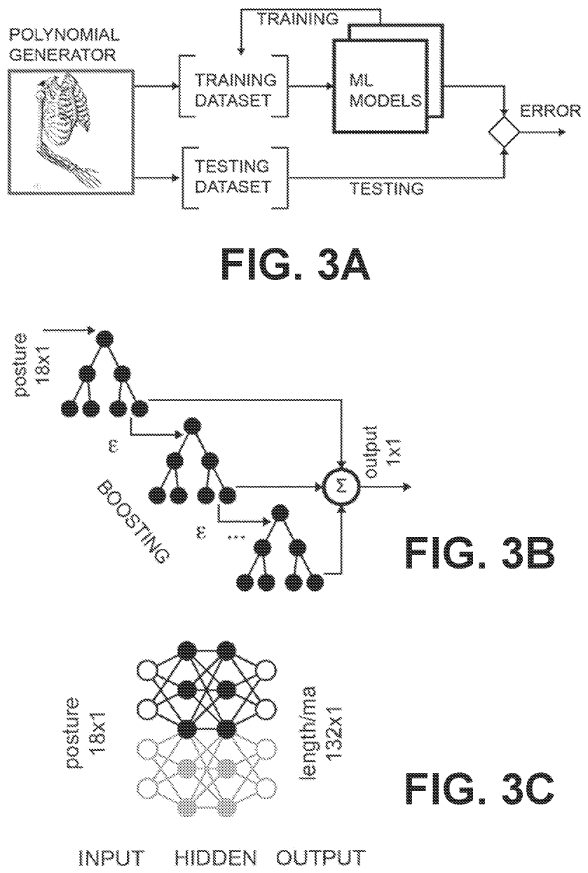

Machine Learning Models. Two types of ML models can be used to map the musculoskeletal input-output relationships. Light gradient boosting machine (LGB) models and artificial neural network (ANN) with two hidden layers were used. The models were trained and tested according to the workflow in FIG. 3A. As illustrated in the example, the polynomial generator can create reference datasets, which can then be iteratively used for training and testing. Validation can accompany the training process to prevent overfitting.

Light gradient boosting machine (LGB): LGB algorithms belong to the group of gradient boosting methods based on choosing iteratively simple learner functions that point to the global minimum in the cost function. Gradient boosting is a technique to assemble weak prediction models (e.g., regression trees) as processing stages that reduce performance errors. The regression trees can use binary recursive decisions to follow a path along hierarchically organized nodes that terminate with the final branches, called leaves. The training process was the search for the optimal routing of inputs so that similar outputs were grouped together.

The boosting method assembles the sequences of multiple regression trees to process errors in stages and gradually improve output accuracy. FIG. 3B illustrates an example of an LGB model with decision binary trees using gradient boosting. The transformation from input postures to output scalar values corresponding to either muscle length or moment arm values was performed in boosting stages to improve accuracy.

Gradient-based one-side sampling in LGB can be used to select a set of inputs where previous weak learner models have the largest output errors. The structure of the decision trees adapted to the needed error tolerance by expanding the number of nodes (leaves) up to the maximal preset value determined empirically. A Microsoft open source implementation of LGB (i.e., lightgbm v.2.2.3, Mircosoft Corp.) was used. The implementation of LGB utilizes multiple parameters for training the model that improves the transformation by adding nodes to trees (leaf-wise tree growth). A set of parameters was kept constant across all models:

import lightgbm
params = {‘seed’: 2523252, ‘tree_learner’: ‘serial’,
‘pre_partition’: True, is_unbalance’:
False, ‘early_stopping_rounds': 200, ‘metric’: ‘mse’}
lightgbm.LGBMRegressor(**params)

The additional training parameters can be added as inputs to the params statement and varied across models. The following is the example implementation for one of the models:

params = {‘seed’: 2523252, ‘tree_learner’: ‘serial’,
‘pre_partition’: True, ‘is_unbalance’:
False, ‘early_stopping_rounds': 200, ‘metric’:
‘mse’,“num_iterations”: 358,
“num_leaves”: 41, “learning_rate”: 0.040600000000000004,
“max_depth”: 18,
“min_data_in_leaf”: 67, “max_drop”: 23, “bagging_fraction”: 0.8,
“feature_fraction”:
0.8 }

Each muscle length and moment arm relationship with posture can be fitted with one LGB model. The full arm and hand model were simulated by 33 length and 99 moment arm transformations of 18-dimensional posture input. Three types of hyper-parameters were iteratively optimized prior to training: 1) the number of leaves in a single decision tree (e.g., a range: 20-100); 2) the minimal number of samples in one leaf (e.g., range: 10-100); and 3) the maximum tree depth as the number of split levels (e.g., range: 1-100). Values for each LGB model can be determined iteratively using the Bayesian optimization on training and validation datasets selected as, e.g., 70% and 30% of all data, respectively. Other hyper-parameters within LGB models, e.g., the number of weak estimators in boosting (e.g., 100), were chosen as defaults of Microsoft implementation v.2.2.3.

Artificial neural network (ANN): Two ANN models can be developed to evaluate posture-dependent muscle lengths and moment arms. Fully connected feed-forward layers with one input, one output, and two hidden layers with rectifying linear units as the outputs of every layer were selected. FIG. 3C illustrates an example of an ANN with two hidden layers performed transformation for all lengths and moment arms in the model. This standard model can provide robust gradient propagation with efficient computation. Using TensorFlow, networks consisting of the following number of nodes in input, two hidden, and output layers: [18, 1024, 512, 33] were composed for the approximation of 33 muscle lengths, and [18, 2048, 1024, 99] for the approximation of 99 moment arms.

Xavier initialization method can be used to select the initial weights for each layer from the normal distribution with zero mean and its variance as 2/(nᵢₙ+nₒᵤₜ), where nᵢₙ and nₒᵤₜ were the number of inputs and outputs in this layer. The network was trained with the batches of sample data (256 samples) using a gradient based stochastic optimization method minimizing a custom cost function. A cost function that focused on the performance of the worst approximations evaluated as RMSE of the worst 5% of input-output pairs from each muscle was developed. The scalar cost was evaluated as the mean of all errors within the upper 30% range.

The variable learning rate can be used to improve the learning dynamics. For example, the initial rate of 0.001 was reduced by 20% if the measured metric stopped improving after two full training dataset evaluations, or epochs. Additional two manipulations to improve learning have been tested. For instance, the variation of processing structure to improve the generalization of solutions distributed across multiple nodes in the ANN was tested. The model was trained with 50% of the nodes skipped in each evaluation and temporarily and randomly assigned to the dropout layer. In addition, the normalization of input samples has been tested. However, the improvements due to the additional structure variation and the normalization were marginal, and it was decided to exclude these manipulations from the processing pipeline.

The presence of overfitting in training was assessed by tracking the divergence in the error rates for training (observed) and testing (unobserved) samples. The difference in errors was less than 0.4% for all muscles without the divergence. For example, for as little as 1000 samples, the RMSE of the trained model for Pronator Teres length was 78.69% for the training set and 79.02% for the testing set, which indicated the absence of overfitting in the early stage of fitting. The difference between training and testing evaluations remained below 0.1% until the terminal level was achieved.

### Examples & Results

Two types of ML models were trained to approximate the musculoskeletal

relationships. The findings detail the training outcome and the training dynamics for learning the transformation from joint posture to muscle lengths and moment arms.

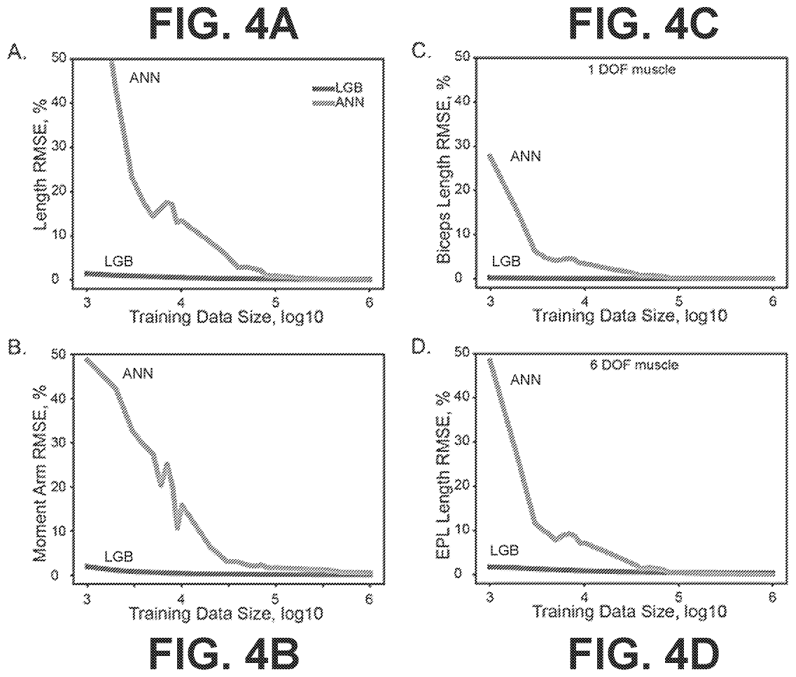

Estimation of the training dataset size: The selection of the training dataset size for ANN and LGB models is a non-trivial step in the model development. The source of data was expressed functionally allowing unlimited source of training data. However, the selection of an optimal dataset that captures the relationships without the tendency for overfitting was one goal of the development. An RMSE metric was used for both length and moment arm models trained with several datasets of incremental size. The relationship between the metric and the dataset size are shown in FIGS. 4A-4D. These plots illustrate examples of the relationship between model performance (RMSE) and the training dataset size for the ANN and LGB models. Examples of the length and moment arm relationships with the size are shown for all muscles in FIGS. 4A an 4B, respectively. Selected examples of muscle length and the dataset size are shown for Biceps Brachii Long Head and Extensor Pollicis Longus in FIGS. 4C and 4D, respectively.

As the size of the dataset increased logarithmically (from 10³ to 10⁶ samples), the training accuracy also increased, with minor improvement in the range above 10⁵ samples. The improvements with the dataset size were not as pronounced showing 1.45% and 1.94% errors with the smallest datasets (10³ samples). The improvement curve of LGB is flat, showing no further improvement, after 10⁵ sample size. The performance of the relatively simple (Biceps Brachii Long Head) and complex (Extensor Pollicis Longus) muscles is illustrated in FIGS. 4C and 4D. These two muscles are on the opposite extremes of complexity expressed as the number of terms needed for an accurate polynomial fit. The improvements are qualitatively similar for both of these muscles indicating no strong dependency on muscle path complexity with both types of models. The largest dataset size was used for all further model development described below.

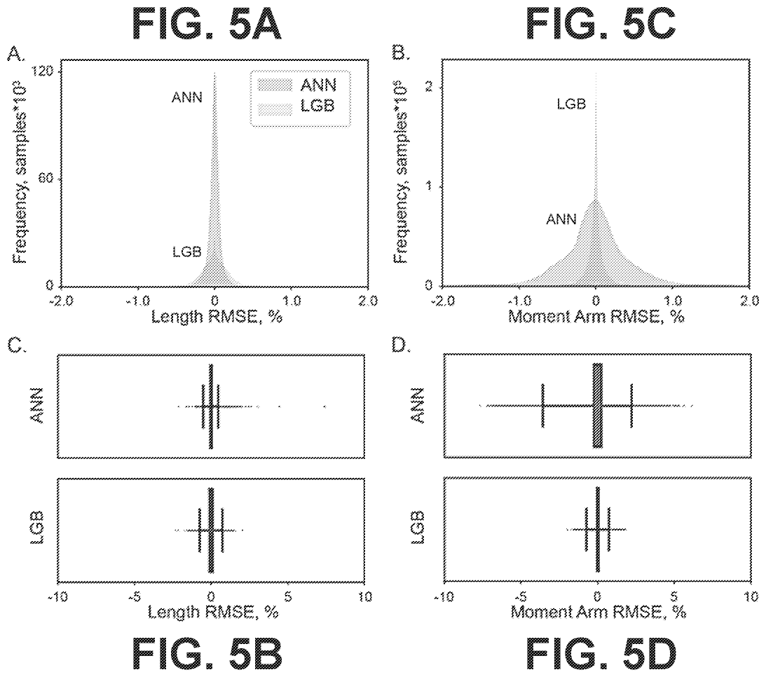

Model accuracy: High accuracy was achieved with both LGB and ANN model types. The distribution of errors is shown in FIGS. 5A-5D with the histograms of RMSE values for the testing dataset (5*10⁴ samples). FIGS. 5A and 5B illustrate the distribution of normalized errors in the prediction of muscle length and moment arms, respectively, for the two ML models, LGB and ANN. The distribution of all errors including all the outliers are shown for muscle length and moment arms in FIGS. 5C and 5D, respectively. The box plots indicate 25% to 75% IQR with whiskers set to cut ±0.1% of the distribution. The highest achieved performance of ANN models was with 10⁷ with absolute errors at 0.08±0.05% for muscle lengths and 0.53±0.29% for moment arms. Similarly, LGB models generated accurate predictions with large training datasets, 0.18±0.06% and 0.13±0.07% errors, respectively. This is the expected error rate based on the previous analysis.

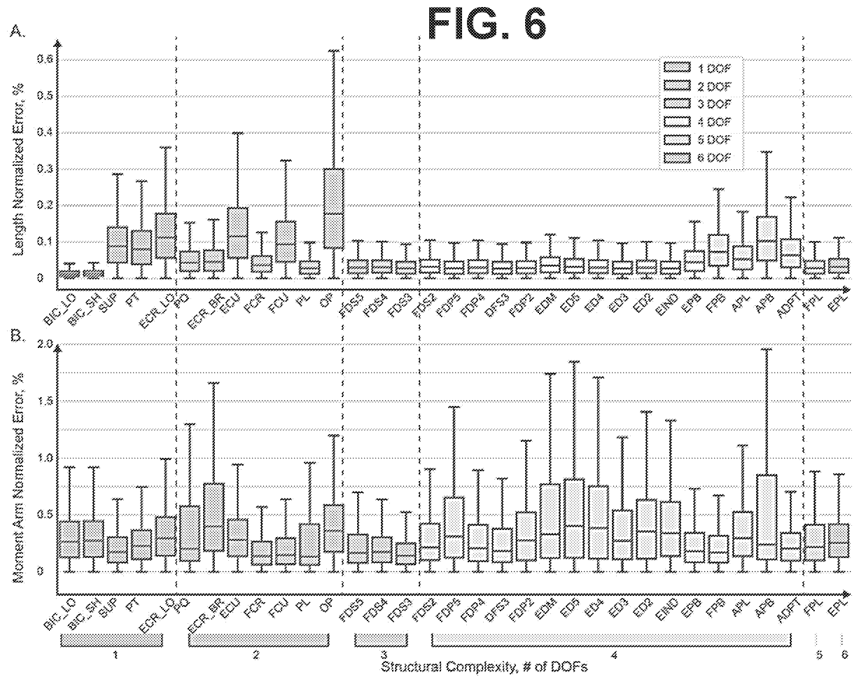

Overall, the error span did not exceed 0.6% for muscle lengths and 2% for muscle moment arms, shown in FIGS. 5A-5D and FIG. 6, which illustrates an example of the normalized absolute errors for muscle length (upper distributions—A) and moment arms (lower distributions—B) evaluated with the ANN. The box plots indicate 25% to 75% IQR with whiskers set to cut 1.5% of the distribution. No relationship was found between length and moment arm errors (p=0.746,R²=0.003). The level of errors was comparable for both simple and complex muscles (spanning more than 3 DOF), e.g., the errors of ECR_BR (a two DOF muscle) were comparable to those of EDM.

The accuracy of LGB and ANN models was comparable. The interquartile ranges (IQR), corresponding to the distance between 25% and 75% level for the distribution of all length error values were 0.075% (ANN) and 0.216% (LGB). The 25-75% IQRs for moment arm errors in FIG. 5B were 0.464% (ANN) and 0.0782% (LGB). Median RMSE values for all models were less than 0.01%. To check if accuracy declined in the extreme postures (1st and 4th quartiles), testing was repeated for errors and a similar rate of errors was found, 0.144% and 0.587% for lengths and moment arms.

The distribution of absolute errors is shown for each muscle in FIG. 6. The muscles are sorted according to the number of DOFs they actuate with relatively simple muscles on the left and complex (thumb) muscles on the right. Overall, the majority of distributions were below 0.2% for 75% of all values, as indicated by the top value of the interquartile range in the box plots. The largest length errors were observed in OP, which was also one of the most difficult muscles to design structurally. In this muscle the top value (Q3) of the interquartile range was about 0.3%, which corresponds to the error of 0.018 mm. In the upper distribution (A) in FIG. 6, the normalized errors of muscle length are presented for each muscle. For all muscles, most errors (up to 75% of the distributions) are below 0.2%. The prominent exception is OP with the highest normalized errors, which is explained by the minimal full physiological range of only 6 mm. The error of 0.3% in OP length corresponds to the absolute error of 0.018 mm. The errors did not increase with muscle structural complexity. The evaluation errors in muscle length where generally larger in the group of muscles spanning 2 DOF (e.g., Extensor Carpi Radialis, ERC_LO) and were comparable to the errors in complex muscles (e.g., Abductor Pollicis Brevis, APB).

Training and evaluation time: The execution times were compared for ANN and LGB models (1.4 GHZ Quad-Core 8th-generation Intel Core i5) by measuring the duration of 1000 evaluations (using method time from the standard time library in Python 3.7). For a given posture, ANN models evaluated both muscle length and moment arms with the combined latency of 1.1±0.6 ms, as compared to 43.1±8.3 ms for LGB models, which were about 39 times slower.

In this disclosure, a solution to the musculoskeletal kinematics problem over the full physiological range of limb postures using ML approaches has been presented. Two standard types of models-LGB and ANN-that both accomplished the mapping from limb posture to muscle kinematic state described by multidimensional muscle length and moment arms were presented. The LGB and ANN approaches were chosen as ML equivalents to the phenomenological model developed to approximate posture dependent muscle parameters with polynomial structures. Both ML methods produced close approximations with the best results achieved by the ANN approach (RMSE=0.08%) as compared to the LGB approach (RMSE=0.12%) for moment arms. LGB and ANN methods have not been previously demonstrated for the solution of the musculoskeletal kinematics.

Motor intent decoding: Estimating limb posture from EMG in real-time applications remains a challenge in human-machine interfaces due to: 1) the difficulty in the theoretical description and 2) the lack of experimental data to validate these models. In general, a statistical mapping between posture and recorded activity from descending pathways, nerves, and muscles has been used as the transformation to predict motor intent, to investigate interplay of mechanical and neural components in pathologies, or to control powered prosthetic limbs or exoskeletal devices. However, the accuracy of decoding realistic movements remains a challenge especially for movements that require dexterous object manipulation.

Many current decoding methods in brain-computer interfaces assume that neural activity is related to limb end-point position and/or velocity and lack the description of movement kinetics generated by muscle forces. The resulting movements of prosthetics are typically slower and less robust than natural movements. Up to five to six arm and hand DOFs can be simultaneously controlled using nerve signals recorded with penetrating electrodes. Accurate but slow movements can be generated for high-dimensional artificial hands with wrist and digits; however, the accuracy is challenged by changing limb posture and orientation. One potential solution is the use of closed-loop control systems that provide not only the forward control of prosthetic, but also incorporate the sensory feedback within neuroprosthetics. A closed-loop control system that takes into account muscle forces would depend on the accurate representation of musculoskeletal actions as described.

The generalizable control solutions based on the biomechanical transformations have potential advantages over the nontransparent statistical approaches. Mechanistic musculoskeletal models of legs and arms may be used for motor intent decoding. The major advantage of biomechanical modeling over statistical methods is in the explicit representation of kinematic and kinetic dependencies in the generated motor command signals during multisegmented limb movements. Thus, the transformation from the recorded biological signals to the proportional control of limbs should be intuitive, given that the underlying computations are sufficiently accurate and without extensive processing delays.

This motivated the exploration and rationale for developing accurate ML methods of approximating the musculoskeletal transformations with physics-informed neural networks. A model driven training and testing of ML algorithms can be used to approximate posture-dependent changes in muscle lengths and moment arms of distal arm and hand muscles. The developed polynomial model can provide a functional representation of data across all possible limb postures. Since any volume of data could be generated, the extent of data needed to train ANN and LGB models was tested. FIGS. 4A-4D illustrate the expected inverse relationship between the approximation errors and the training dataset size. Using only 10⁶ samples for training ANN and LGB models resulted in kinematic errors that were less than 0.5%. This number of samples is about one order of magnitude lower than the number of samples required for previous implementations using an information theory-based algorithm for approximating the same kinematic variables. This may indicate that ANN and LGB methods may further generalize the polynomial relationships that needed a larger volume of samples to generate functional muscle-posture representations limited to polynomial power terms.

Computational delays: The transformation of EMG signals into movement rarely accounts for the musculoskeletal anatomy and physiology. This is partly due to the extreme complexity of muscle organization and nonlinear intrinsic muscle properties that include independent force-length and force-velocity relationships and less popular in modeling, short-range stiffness, which is a hysteretic force-length property. The task of simulating muscle force generation requires adequate structural information about muscle paths and posture-dependent changes in moment arms. The development of complex musculoskeletal models can be simplified by the dedicated simulation tools for editing and simulating segmental dynamics—OpenSim, MuJoCo, Simscape. The challenge remains in collating sufficient datasets of musculoskeletal measurements for creating complex musculoskeletal models and then in testing and validating these models across the full-range of motion to ensure their use in a wide range of applications.

The computational delays of solving the equations of motion governing limb dynamics have previously impeded the application of model-based prosthetic controllers. In particular, the evaluation of muscle force-velocity characteristic may require sub-millisecond latencies to decode accurately rapid movements computed by a physical engine, which requires additional time to execute (˜1 ms). The disclosed implementations demonstrated a clear speed advantage of the ANN over the LGB model (about 39× faster) and needed about 1 ms on standard hardware. This performance was still about 15 times slower than the polynomial approximation (about 60 μs); yet, it provides a milestone due to the rapid development of both software and hardware solutions for ANNs. Further improvements in the performance of the ANN may be possible approaching the latencies appropriate not only for the feedforward computations, but also for predictive inverse computations inspired by theoretical neural transformations. Another biomimetic feature of the ANN model is its potential solution for increased computational complexity to accommodate the increase in the size of the described structure, typically termed as “the curse of dimensionality”. It has been demonstrated that the typical exponential increase could be replaced with the linear increase in the number of terms needed within the polynomial approximations. Here, the same structure of the ANN model accommodated accurate calculations for a subset and for the full set of 33 muscles. As long as the additional simulated muscles are relatively similar in anatomical complexity to the muscles represented in the current model, the same structure of the ANN should be able to embed their dynamics without an increase in the number of nodes in each layer.

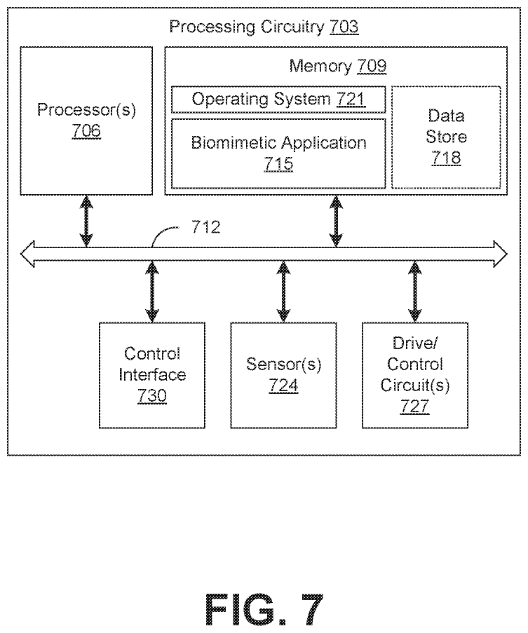

Referring to FIG. 7, shown is an example of processing circuitry 703 that can be utilized in sensorimotor mechanisms (e.g., an artificial limb). The processing circuitry 703 can include at least one processor circuit, for example, having a processor 706 and a memory 709, both of which are coupled to a local interface 712. The local interface 712 may comprise, for example, a data bus with an accompanying address/control bus or other bus structure as can be appreciated. Stored in the memory 709 are both data and several components that are executable by the processor 706. In particular, stored in the memory 709 and executable by the processor 706 are biomimetic application 715 and potentially other applications. It is understood that there may be other applications that are stored in the memory 709 and are executable by the processor 706 as can be appreciated. Where any component discussed herein is implemented in the form of software, any one of a number of programming languages may be employed such as, for example, C, C++, C #, Objective C, Java®, JavaScript®, Perl, PHP, Visual Basic®, Python®, Ruby, Flash®, or other programming languages.

Also stored in the memory 709 may be a data store 718 and other data. The data stored in the data store 718, for example, is associated with the operation of the sensorimotor mechanisms. For example, the data store 718 can include operational parameters, user preference setting parameters, and other data or information as can be understood. In addition, an operating system 721 may be stored in the memory 709 and executable by the processor 706. A number of software components can be stored in the memory 709 and are executable by the processor 706. In this respect, the term “executable” means a program file that is in a form that can ultimately be run by the processor 706. Examples of executable programs may be, for example, a compiled program that can be translated into machine code in a format that can be loaded into a random access portion of the memory 709 and run by the processor 706, source code that may be expressed in proper format such as object code that is capable of being loaded into a random access portion of the memory 709 and executed by the processor 706, or source code that may be interpreted by another executable program to generate instructions in a random access portion of the memory 709 to be executed by the processor 706, etc. An executable program may be stored in any portion or component of the memory 709 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The processing circuitry 703 can monitor the system conditions through one or more sensor(s) 724 (e.g., neuroprosthetic sensor(s), proximity sensor(s), displacement sensor(s), pressure/force sensor(s), etc.) and provide control signals to various drive and/or control circuitry 727 as has been described. The processing circuitry 703 can interface with a user of the flatbread machine through the control interface 730 to accept inputs and provide. To this end, the control interface 730 can be configured to indicate, e.g., system status and/or prompt for user inputs. The processing circuitry can also be configured to allow for communication with an external device though a communication link or other network connection. For example, a smartphone app that connects to the processing circuitry 703 via Bluetooth®, WiFi, or other appropriate communication link. The ability to communicate through the communication link or network connection also allows for downloading and/or updating the firmware and/or software (e.g., through the smartphone app), and upload and/or transfer operational data to support resources such as a website.

Although the biomimetic application 715 and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the biomimetic application 715, that comprises software or code can be embodied in any non-transitory computer-readable medium for use by or in connection with an instruction execution system such as, for example, a processor 706 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

Deep learning is a relatively new computational technique for the description of the musculoskeletal dynamics. The experimental relationships of muscle geometry in different postures are the high-dimensional spatial transformations that can be approximated by relatively simple functions, which opens the opportunity for machine learning (ML) applications. In this study, general ML algorithms were challenged with the problem of approximating the posture-dependent moment arm and muscle length relationships of the human arm and hand muscles. Two types of algorithms, light gradient boosting machine (LGB) and fully connected artificial neural network (ANN), were used in solving the wrapping kinematics of 33 muscles spanning up to six degrees of freedom (DOF) each for the arm and hand model with 18 DOFs. Using the two ML methods for solving the posture-dependent changes in the musculoskeletal properties for the description of limb kinetics has been demonstrated. The achieved execution accuracy was adequate with both ANN and LGB models and similar to the original polynomial model. ANN model was 39 times faster than LGB model computing muscle variables in 1.1 ms, which is appropriate for real-time control solutions.

The input-output training and testing datasets, where joint angles were the input and the muscle length and moment arms were the output, were generated by our previous phenomenological model based on the autogenerated polynomial structures. Both models achieved a similar level of errors: ANN model errors were 0.08±0.05% for muscle lengths and 0.53±0.29% for moment arms, and LGB model made similar errors—0.18±0.06% and 0.13±0.07%, respectively. LGB model reached the training goal with only 10³ samples, while ANN required 10⁶ samples; however, LGB models were about 39 times slower than ANN models in the evaluation. The sufficient performance of developed models demonstrates the future applicability of ML for musculoskeletal transformations in a variety of applications, such as in advanced powered prosthetics.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

This research was sponsored by the U.S. Army Research Office and the Defense Advanced Research Projects Agency (DARPA) under Cooperative Agreement Number W911NF-15-2-0016. The views and conclusions contained in this document are those of the authors and should not be interpreted as representing the official policies, either expressed or implied, of the Army Research Office, Army Research Laboratory, or the U.S. Government. The U.S. Government is authorized to reproduce and distribute reprints for Government purposes notwithstanding any copyright notation hereon.

1. A method, comprising:
generating muscle model parameters by a musculoskeletal kinematic transformation implemented by a first artificial neural network, the muscle model parameters based at least in part upon sensor inputs;
generating one or more physics engine parameters from a muscle model, the one or more physics engine parameters based at least in part on the muscle model parameters; and
generating a physics engine transformation implemented by a second artificial neural network based at least in part upon the one or more physics engine parameters, the physics engine transformation representing segment dynamics and interactions with environment.

2. The method of claim 1, comprising controlling a sensorimotor mechanism based upon the physics engine transform.

3. The method of claim 1, wherein the muscle model parameters comprise muscle and joint parameters.

4. The method of claim 3, wherein the muscle and point parameters comprise a plurality of muscle lengths and a plurality of moment arms.

5. The method of claim 1, wherein the physics engine parameters comprise joint torque.

6. The method of claim 5, wherein the physics engine parameters further comprise neural activity.

7. The method of claim 1, wherein the sensor inputs comprise surface electromyography signals.

8. The method of claim 1, wherein training datasets for the first artificial neural network of the musculoskeletal kinematic transformation are generated using an approximation of musculoskeletal relationships.

9. The method of claim 8, wherein the first artificial neural network is trained using a supervised learning approach.

10. The method of claim 1, wherein the first and second artificial neural networks have a latency of less than 20 ms.

11. A system for prosthetic control, comprising:
a plurality of sensors; and
processing circuitry configured to control a sensorimotor mechanism based upon a physics engine transformation implemented by a second artificial neural network based at least in part upon one or more physics engine parameters generated from a muscle model, the one or more physics engine parameters based at least in part on muscle model parameters generated by a musculoskeletal kinematic transformation implemented by a first artificial neural network, the muscle model parameters based at least in part upon sensor inputs from the plurality of sensors.

12. The system of claim 11, wherein the plurality of sensors comprises surface electromyography sensors.

13. The system of claim 11, wherein the physics engine parameters comprise joint torque.

14. The system of claim 13, wherein the physics engine parameters further comprise neural activity.

15. The system of claim 11, wherein the muscle model parameters comprise muscle and joint parameters.

16. The system of claim 15, wherein the muscle and point parameters comprise muscle lengths and moment arms.
